(19) United States
(12) Patent Application Publication
(10) Pub. No.: US 2022/0008449 A1
(43) Pub. Date: Jan. 13, 2022

(54) A METHOD OF MIMICKING BENEFITS OF DIETARY RESTRICTION BY TRANSIENTLY UPREGULATING ER STRESS RESPONSE

(71) Applicant: NATIONAL INSTITUTE OF IMMUNOLOGY, New Delhi (IN)

(72) Inventors: Arnab MUKHOPADHYAY, New Delhi (IN); Kausik CHAKRABORTY, New Delhi (IN); Latika MATAI, New Delhi (IN)

(73) Assignee: NATIONAL INSTITUTE OF IMMUNOLOGY, New Delhi (IN)

(21) Appl. No.: 17/294,082

(22) PCT Filed: Dec. 13, 2019
(86) PCT No.: PCT/IN2019/050917

§ 371 Date: May 14, 2021

(30) Foreign Application Priority Data
Dec. 14, 2018 (IN) 201811047507

(51) Int. Cl. A61K 31/7072; A61K 31/7004; G16H 20/60

(52) U.S. Cl. CPC A61K 31/7072; G16H 20/60; A61K 31/7004

(57) ABSTRACT

A role of molecular mechanisms determine the longevity of an organism, particularly, method of mimicking benefits of dietary restriction by transiently upregulating endoplasmic stress response.

The following specification particularly describes the invention and the manner in which it is to be performed

### Technical Field

The present disclosure is generally related to the field of molecular biology. Specifically, the present disclosure provides a method of increasing lifespan by restricting diet which improves proteostasis in a subject. More specifically, the present disclosure provides a method of inducing an early and transient upregulation of the Unfolded Protein response (UPR^{ER}) in a subject by either generating a nutrient restriction or administering pharmaceutical reagent. Also, the present disclosure provides a method of treating protein folding disorders.

### Background Art

A vast majority of the secreted as well as membrane proteins fold and mature in the endoplasmic reticulum (ER) before they are exported to their destinations. The protein folding capacity of the ER is carefully monitored and calibrated by three conserved signal transduction pathways, collectively called the Unfolded Protein Response (UPR^{ER}) regulators, that ensure protein homeostasis (proteostasis) and proper cellular function. This is achieved by activating the proteostasis network (PN) consisting of molecular chaperons, protein degradation machinery and stress response pathways that act to resolve consequences of protein misfolding in the ER. In metazoan ER, the three arms of UPR^{ER}, namely IRE1, PERK and ATF6 function in parallel to trigger the PN and counteract ER stress by a) increasing folding capacity through the expression of various chaperons, b) attenuating translation to reduce protein load in ER, c) regulated IRE1-dependent decay of mRNA (RIDD), or d) activating ER-associated proteasomal degradation (ERAD) to remove terminally misfolded proteins.

Aging is characterized by a catastrophic collapse of the proteostasis network due to the loss of protein quality control machineries across the cellular compartments. With age, the ER structure begins to deteriorate, and the ER health fails as it is unable to mount an optimal UPR^{ER}.

Expression levels of key ER mRNA and proteins as well as activities of ER resident proteins, including BiP, PDI, calnexin and GRP94 are known to decline with age. As a possible consequence, many diseases of protein misfolding including Alzheimer's, Parkinson's and Huntington's diseases have an age-onset.

The flagging cellular proteostasis can be improved simply by exposing cells or organisms to moderate stress before they encounter acute stress. This process is called hormesis and has been shown to be beneficial to life span and health.

Hormesis can be affected in a compartment-specific manner in a cell. For example, in C. elegans, Drosophila and human fibroblasts, hormetic heat shock upregulates cytosolic molecular chaperones that further protect against acute heat stress. In C. elegans, glucose restriction increases mitochondrial respiration and ROS production, which results in protection against oxidative stress and enhanced longevity, a process termed mitohormesis. Mitohormesis has also been implicated in other long-lived mutants in worms and fly. Similarly, ER hormesis has been observed in Drosophila carrying mutations associated with ER protein folding/degradation machinery that induce cytoprotective response towards subsequent ER insults. In mammalian cells, ER stress preconditioning protects against brain ischemia and attenuates heart ischemia/reperfusion injury. ER preconditioning is also neuroprotective in Drosophila and mice models of Parkinson's disease. The mutation in the C. elegans heterochromatin protein like-2 (HPL-2), induces hormetic induction of stress resistance dependent on the IRE-1 branch of UPR^{ER}. Further, expression of xbp-1s in neurons has been shown to increase longevity in a cell non-autonomous manner, suggesting a possible role of ER hormesis in life span regulation.

Dietary Restriction (DR) is a conserved intervention that can delay proteostasis, activate cytoprotective pathways and increase life span across species. A wealth of evidence suggests that dietary restriction may act as a mild stress that enhances lifespan through hormesis. The fact that glucose restriction that is akin to DR increases life span through mitohormesis attests to this. However, the involvement of ER hormesis in DR is not known.

The present invention shows that dietary restriction transiently upregulates UPR^{ER} during early larval development. Mimicking this response with a pharmacological glycosylation inhibitor, tunicamycin or a non-hydrolysable glucose analog, increases life span. Both transient UPR^{ER} and increased life span depend on the ER stress sensor, IRE-1.

The present application shows that the transient ER stress leads to better iUPR^{ER} at adulthood and better proteostasis. This hormetic dose of ER stress during development upregulates ERAD genes leading to efficient degradation of ER resident proteins and thus, contributes towards enhanced proteostasis.

### SUMMARY

The present disclosure is related to a method of inducing an early and transient upregulation of Unfolded Protein Response (UPR^{ER}) in a subject by either generating a nutrient restriction or administering pharmaceutical reagent, wherein said method mimics the pro-longevity effects of the dietary restrictions (DR). It is shown in the present invention that a transient pharmacological ER stress, if imposed early in development on Caenorhabditis elegans, enhances proteostasis, prevents iUPR^{ER} decline with age, and increases adult life span Importantly, dietary restriction (DR), that has a conserved positive effect on life span, employs this mechanism of ER hormesis for longevity assurance.

### BRIEF DESCRIPTION OF DRAWINGS

The disclosure may be more fully understood by reading the following detailed description of the embodiment, with reference made to the accompanying drawings as follows:

FIG. 1: DR triggers a transient up-regulation of UPR^{ER} early in life. (A) Representative images of eat-2(ad1116);Phsp-4:gfp(zcIs4) worms at 34 hours post-bleaching as compared to the Phsp-4:gfp(zcIs4) worms. Densitometric quantification averaged over three biological repeats, on right, shows the transient upregulation of basal UPR^{ER}. Error bars-SEM, *p<0.05 as determined by student's t test. (B) Representative images of Phsp-4:gfp(zcIs4) and eat-2(ad1116);Phsp-4:gfp(zcIs4) at indicated larval stages and in gravid adults. Densitometric quantification (right panel) shows no upregulation in basal UPR^{ER} in these stages in eat-2(ad1116). Basal UPR^{ER} is downregulated in eat-2(ad1116) in gravid adults. Average of 2 biological replicates. Error bars-SD, *p<0.05 by student's t test. (C) Representative images of transient UPR^{ER} of Phsp-4:gfp(zcIs4) and eat-2(ad1116);Phsp-4:gfp(zcIs4) grown on control or ire-1 RNAi. Densitometric quantification below shows the upregulation of UPR^{ER} on control RNAi but not on ire-1 RNAi. Average of 2 biological replicates. Error bars-SEM, *p<0.05 by Two-way Annova. (D) Representative images of transient UPR^{ER} of Phsp-4:gfp(zcIs4) and eat-2(ad1116);Phsp-4:gfp(zcIs4) grown in presence or absence of 2% glucose. The transient UPR^{ER} is suppressed in presence of glucose in eat-2(ad1116);Phsp-4:gfp(zcIs4). Quantification is shown on right. Average of 3 biological replicates. Error bars-SEM, *p<0.05 by Two-way Annova. Ns—non-significant. (E) Concavalin A western analysis of WT or eat-2(ad1116) with or without 2% glucose supplementation. WT has more glycosylated proteins compared to eat-2(ad1116). Glucose supplementation increases the levels of glycosylated proteins.

FIG. S (supplementary) 1: (A) Representative images of Phsp-4:gfp(zcIs4) worms, grown on control or drl-1 RNAi, at 32 hours post-bleaching. Densitometric quantification averaged over 2 biological repeats, shows the transient upregulation of basal UPR^{ER}. Error bars-SEM, *p<0.05 as determined by student's t test. (B) Representative images of Phsp-4:gfp(zcIs4) worms growing on control or drl-1 RNAi at indicated larval stages and in gravid adults. Densitometric quantification showing drl-1 knockdown worms having no increase in basal UPR^{ER} response as compared to control RNAi in these stages. Basal UPR^{ER} is lower in drl-1 RNAi worms at gravid adult stage. Average of 2 biological replicates. Error bars are SD. *p<0.05 by student's t test. (C) drl-1 knockdown does not affect cytosolic heat shock response (HSR; upper panel). Representative images of Phsp-16.2:gfp(dvIs70) worms, representing HSR, grown on control or drl-1 RNAi at 32 hours post-bleaching. (lower panel) drl-1 knockdown does not affect mitochondrial stress response (lower panel). Representative images of Phsp-6:gfp(zcIs9) worms, representing UPR^{mt}, grown on control or drl-1 RNAi at 32 hours post-bleaching. Densitometric quantification averaged over 2 biological repeats do not show any difference between drl-1 knockdown and control RNAi, ns-not significant. (D) UPR^{ER} up-regulation in L2 larval stage on drl-1 knockdown is dependent on ire-1. Representative images of Phsp-4:gfp(zcIs4) and ire-1(zc14);Phsp-4:gfp worms growing on control or drl-1 RNAi at 32 hours post-bleaching. Densitometric quantification of GFP fluorescence averaged over 2 biological repeats is shown in the lower panel. *p<0.05 as determined by Two-way Annova.

FIG. 2: ire-1 is required for longevity assurance by DR and transient Tm supplementation. (A) Lifespan extension in eat-2(ad1116) mutant is suppressed on knocking down ire-1. (B) Atf-6 knockdown has little effect on eat-2(ad1116) lifespan (C) Pek-1 knockdown has no effect on lifespan extension in eat-2(ad1116) mutant. (D) Transient supplementation of Tm during initial larval stage is sufficient to extend lifespan. WT worms were bleached, and eggs were added to M9 buffer containing bacterial feed and supplemented with 0.125 μg/ml Tm for the first 24 hours and later scored for adult lifespan. As a control, worms were treated with 0.05% DMSO, (Tm=0 μg/ml). (E) Tm-mediated increase in lifespan is dependent on ire-1. WT and ire-1(v33) mutants were supplemented with Tm (0.125 μg/ml) for initial 24 hours and later scored for adult lifespan. (F) Tm supplementation cannot further increase lifespan of eat-2(ad1116). Complete life span data is present in Table S1.

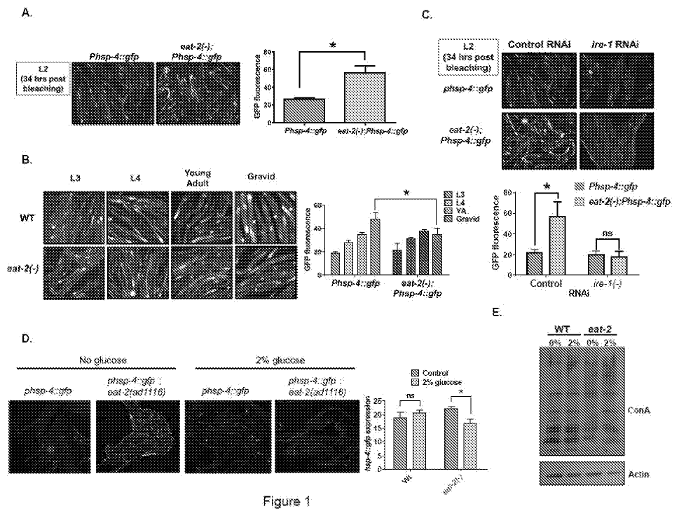

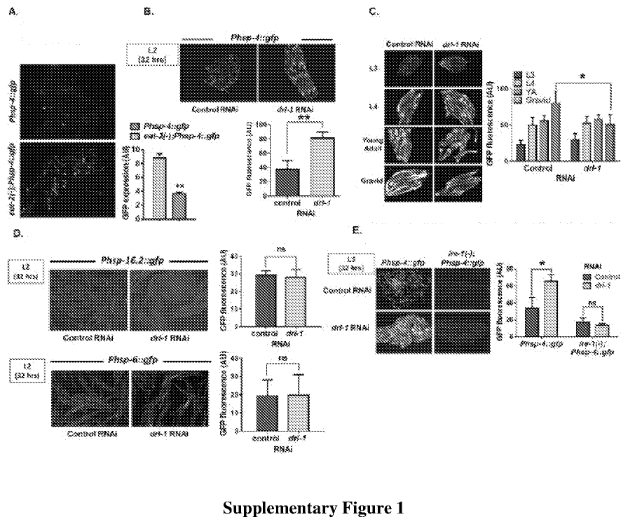

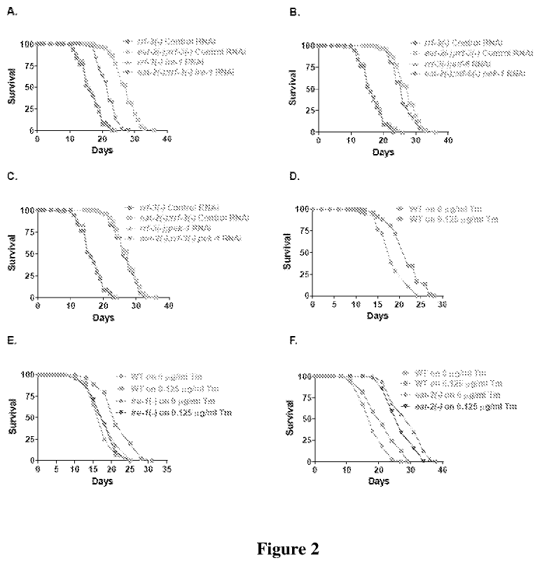

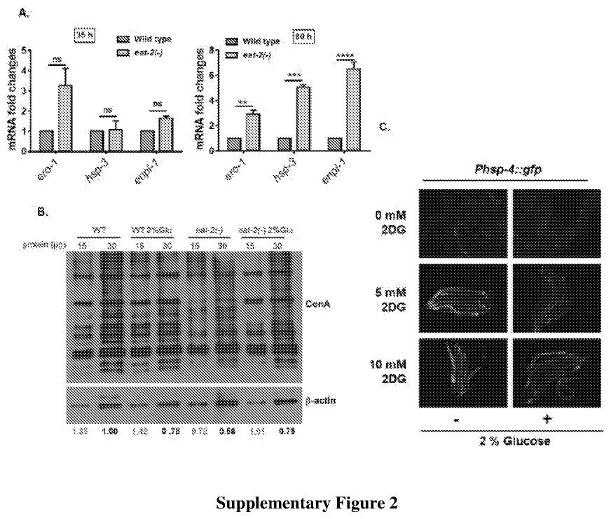

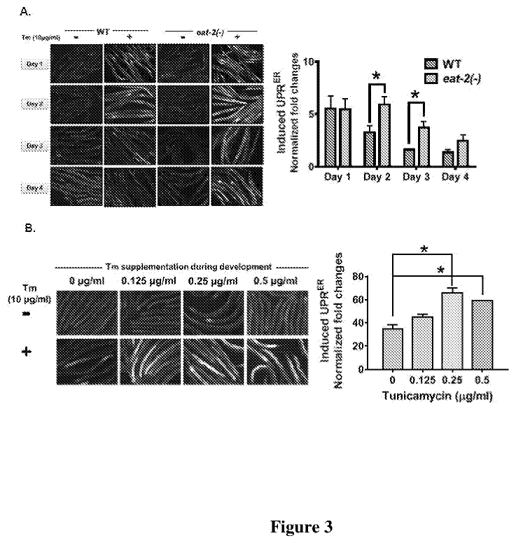

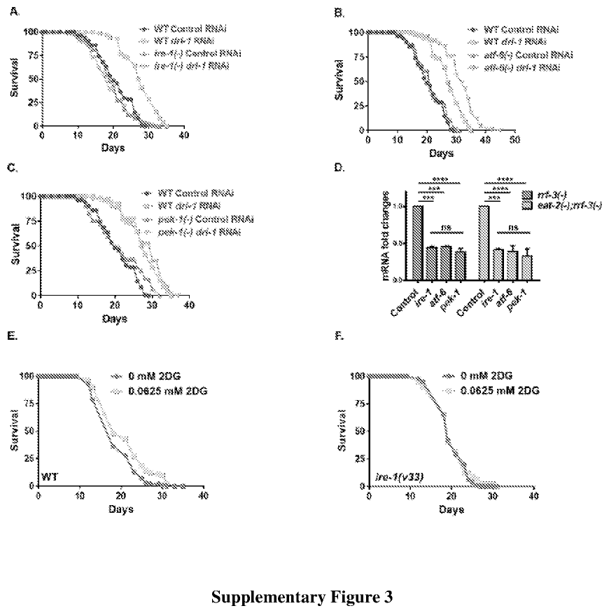

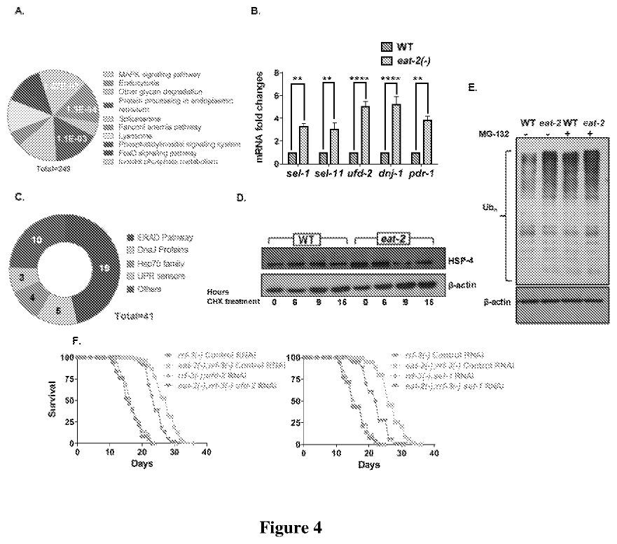

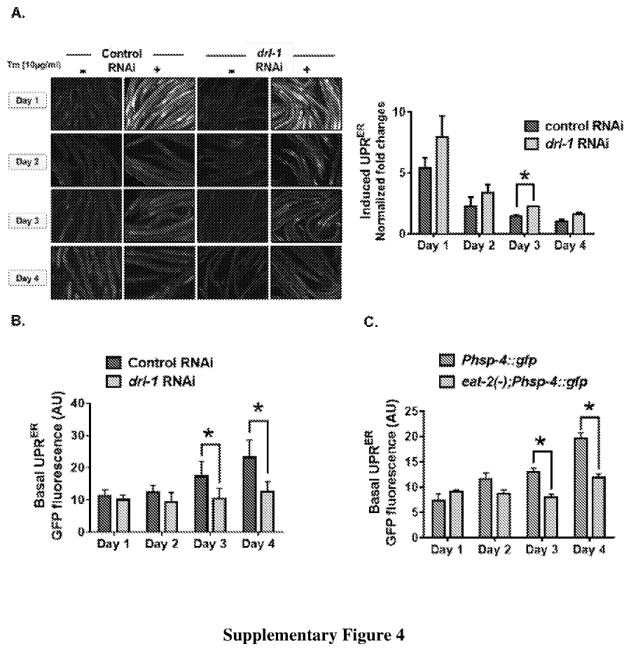

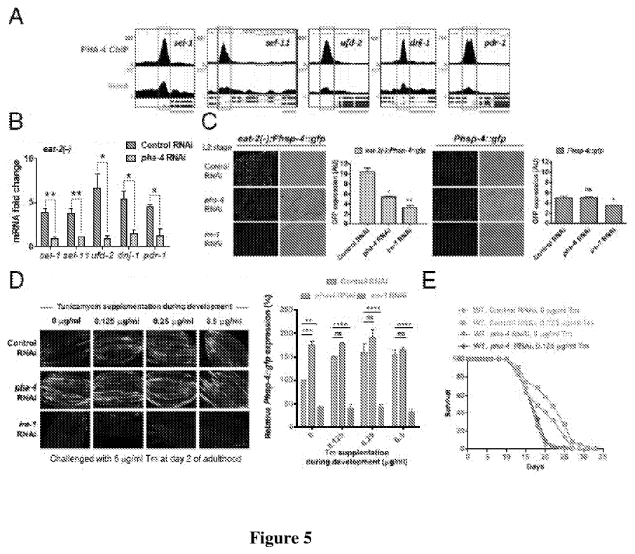

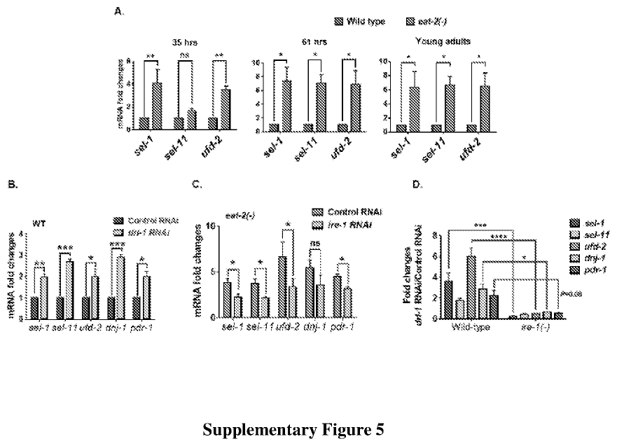

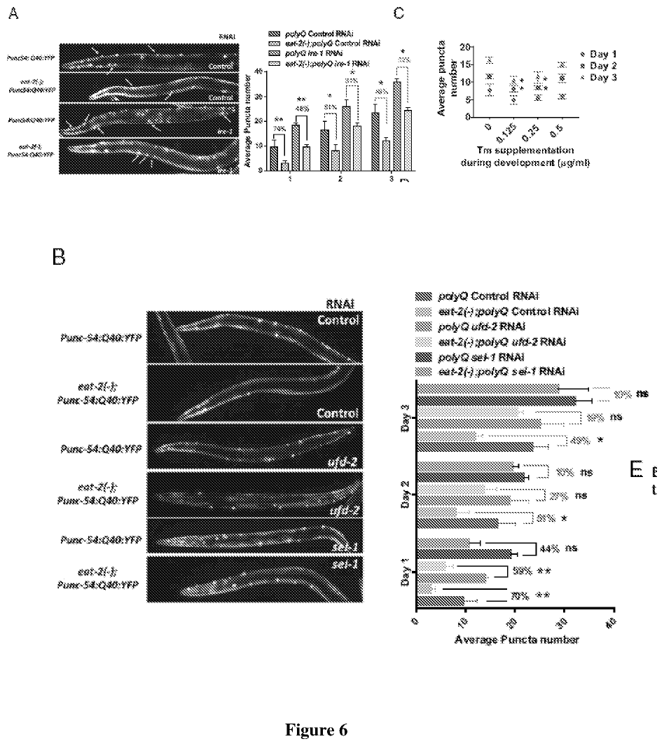

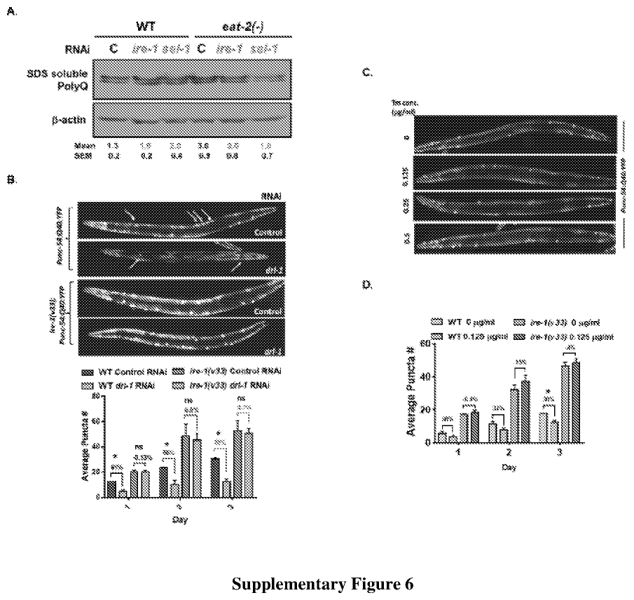

FIG. S2: (A) The expression of ER resident proteins is upregulated in eat-2(−) as compared to WT during different stages of post-embryonic development, determined by QRT-PCR analysis. Average of 3 biological replicates. Error bars—SEM, **p<0.01, *** p<0.001, **** p<0.0001, Student's t test. (B) Concavalin A western blot analysis was performed after loading different concentrations of WT or eat-2(−) (with or without exposure to 2% glucose) proteins. The bands were quantified after normalizing with β-actin. Supplementary data of FIG. 1E. (C) Phsp-4:gfp(zcIs4) worms were exposed to different concentrations of 2-Deoxyglucose (2DG) with or without 2% glucose. Glucose supplementation reduced upregulation of hsp-4::gfp at L2 stage in worms treated with 2DG.

FIG. 3: DR delays age-associated reduction in induced-UPR^{ER} (iUPR^{ER}) efficiency. (A) eat-2(ad1116) mutants have delayed age-associated decline in iUPR^{ER} efficiency. Representative images of Phsp-4:gfp and eat-2(ad1116);Phsp-4:gfp worms with/without Tm treatment (10 μg/ml for 6 hours) on different days of adulthood. Graph represents normalized fold induction in GFP fluorescence after 6 hours of Tm treatment in WT and eat-2(ad1116) on different days of adulthood, averaged over 3 biological repeats. [Normalization was performed with the basal GFP fluorescence (basal UPR^{ER}) for individual experiment]. (B) Transient Tm supplementation increases iUPR^{ER} efficiency at day 2 of adulthood. Representative images of Day 2 adult Phsp-4:gfp(zcIs4) worms growing on control RNAi, treated with different concentrations of Tm for 24 hours post-bleaching and further treated with Tm (10 μg/ml for 6 hours) at day 2 of adulthood. Graph represents normalized fold induction in GFP fluorescence after 6 hours of Tm treatment on day 2 of adulthood averaged over 2 biological repeats. [Normalization was performed with the fold induction in GFP fluorescence in adults not exposed to Tm earlier]. Error bars are SEM. * represents p<0.05, as determined by student's t test.

FIG. S3: (A) In an ire-1 mutant, drl-1 knockdown fails to extend lifespan. (B-C) Knocking down drl-1 can increase the lifespan of atf-6 (B) and pek-1 mutants (C). (D) Life span of WT is increased on transient supplementation with 2DG. WT worms were bleached, and eggs were added to M9 buffer containing bacterial feed and supplemented with 0.0625 mM 2DG for the first 24 hours and later scored for adult lifespan. As a control, worms were treated with water (2DG =0 mM). (E) Transient 2DG treatment failed to increase life span in ire-1(v33). Complete life span data is present in Table S1.

FIG. 4: ER protein processing machinery is transcriptionally up-regulated in DR adults. (A) Pie-Chart showing KEGG pathway/Gene ontology analysis of the transcripts up-regulated in eat-2(ad1116) worms as compared to wild-type (N2) at day 1 of adulthood. Forty-one transcripts pertaining to protein processing functions in ER were up-regulated, according to the analysis performed using DAVID functional annotation tool. The listed pathways have p<0.05 and FDR<10 (Bonferroni Corrected p value—0.0011). (B) Quantitative real time-PCR (QRT-PCR) analysis to detect the expression of ER protein processing genes reports significant up-regulation in eat-2(ad1116) adults as compared to WT at Day 1. Error bars indicate SEM over 3 independent biological replicates. Error bars indicate SEM. *p<0.05, ** p<0.01,*** p<0.001,**** p<0.0001 as determined by the student's t test. (C) ER-associated Degradation pathway (ERAD) genes are up-regulated in eat-2(ad1116) worms. Pie-chart representing classification of the up-regulated ER genes. Approximately 50% (19 out of 41) of up-regulated genes function in the ERAD. (D) Day 1 adult WT and eat-2(ad1116) worms were treated with cycloheximide (2 mg/ml) for indicated time points and HSP-4 levels were detected by western blot. (E) Ubiquitinated proteins were detected using a polyubiquitin antibody in WT and eat-2(ad1116) adults with or without proteasomal inhibitor, MG132 treatment. (F) DR-mediated longevity is dependent on ERAD genes ufd-2 and sel-1. Lifespan extension in eat-2(ad1116) worms was partially suppressed on knocking down ufd-2 or sel-1 using RNAi. Complete life span data is present in Table S1.

FIG. S4: (A) Drl-1 knockdown delays age-associated decline in iUPR^{ER} efficiency. Representative images of Phsp-4:gfp(zcIs4) worms grown on control or drl-1 RNAi with/without Tm treatment (10 μg/ml for 6 hours) on different days of adulthood. Graph represents normalized fold induction in GFP fluorescence after 6 hours of Tm treatment on different days of adulthood. [Normalization was performed with the basal GFP fluorescence (basal UPR^{ER}) for individual experiment]. (B) Densitometric quantification of GFP fluorescence in control RNAi- or drl-1 RNAi-treated worms without any treatment (Basal UPR^{ER}) on indicated days. (C) Densitometric quantification of GFP fluorescence in WT and eat-2 mutants without any treatment (Basal UPR^{ER}) on indicated days. Error bars are SEM, average of 2 biological replicates, * represents p<0.05, as determined by student's t test.

FIG. 5: PHA-4 regulates diverse aspect of ER homeostasis during DR. (A) University of California, Santa Cruz browser view of PHA-4/FOXA peaks on promoter proximal regions of ER protein processing genes, as determined by ChIP-seq analysis of the unc-119(ed3)III;wgIs37(OP37) strain; data mined from MODENCODE and reanalyzed using our bioinformatic pipeline. Red boxes indicate the promoter regions where peaks are observed. (Upper) PHA-4 ChIP using anti-GFP antibody. (Lower) Input DNA. Light blue bars represent a portion of the gene of interest. (B) QRT-PCR analysis was used to compare ER protein processing genes between eat-2(ad1116) and WT adults grown on control or pha-4 RNAi. Graph shows significant suppression of the up-regulated genes on pha-4 knockdown. Average of 3 biological replicates is shown. (C) Knocking down pha-4 by RNAi prevents transient UPRER up-regulation at L2 (32 h postegg synchronization) in eat-2(ad1116). Ire-1 RNAi is used as control. Quantification shown on Right of each figure. Average of 2 replicates is shown. (D) iUPR^{ER} is deregulated on pha-4 knockdown. Phsp-4:gfp(zcIs4)worms, grown on control, pha-4, or ire-1 RNAi, with or without treatment with hormetic dose of Tm were challenged with 5 μg/mL of Tm at day 2 of adulthood. Quantification is provided on Right side. Average of 2 replicates is shown. Error bars are SEM in all cases, *P<0.05, **P<0.01, ***P<0.001, ****P<0.0001, Student's t test. ns, nonsignificant. (E) The life span of WT worms given a hormetic dose of Tm is dependent on PHA-4. WT worms grown on control or pha-4 RNAi were supplemented with Tm (0.125 μg/mL) for the initial 24 h and later scored for adult life span.

FIG. S5: (A) QRT-PCR analysis to detect the mRNA levels of ER protein processing genes show significant up-regulation in Day 1 WT adults grown on drl-1 RNAi as compared to control RNAi. (B) QRT-PCR analysis was used to compare ER protein processing genes between eat-2(ad1116) and WT adults grown on Control or ire-1 RNAi. (C) QRT-PCR analysis to detect the mRNA level of ER protein processing genes in Day 1 WT and ire-1(v33) adults grown on drl-1 RNAi as compared to control RNAi. (D) QRT-PCR analysis was used to compare ER protein processing genes in eat-2(ad1116) compared to WT at different time points during post-embryonic development. (E) QRT-PCR analysis was used to compare ER protein processing genes between eat-2(ad1116) and WT adults grown on Control or pha-4 RNAi shows significant suppression of the up-regulated genes on pha-4 knock down. Error bars indicate SEM. *p<0.05, **p<0.01, ** *p<0.001,****p<0.0001 as determined by the student's t test

FIG. 6: Suppression of PolyQ aggregation with age in DR is dependent on ire-1. (A) Representative images showing polyQ aggregates in Punc-54:polyQ40:yfp and eat-2(ad1116);Punc-54:polyQ40:yfp worms on control or ire-1 RNAi at day 2 of adulthood. Suppression of polyQ aggregation in eat-2 mutants is abrogated on knocking down ire-1. Graph represents percent reduction in the number of aggregates in eat-2(ad1116);Punc-54:polyQ40 worms as compared to Punc-54:polyQ40:yfp worms on control or ire-1 RNAi, at different days of adulthood. Average percentage reduction over three biological replicates is plotted. (B) Representative images showing polyQ aggregates for Punc-54:polyQ40:yfp and eat-2(ad1116);Punc-54:polyQ40:yfp worms on control RNAi or RNAi against ERAD genes (ufd-2 or sel-1) at day 2 of adulthood. Suppression of polyQ aggregation in eat-2 mutants is abrogated on knocking down ufd-2 and sel-1. Graphs represents percent reduction in the number of aggregates in eat-2(ad1116);Punc-54:polyQ40:yfp worms as compared to Punc-54:polyQ40:yfp on control, ufd-2 and sel-1 RNAi, on different days of adulthood. Average percentage reduction over three biological replicates is plotted. (C) PolyQ aggregates in Punc-54:polyQ40:yfp worms on control RNAi that have been exposed to different concentrations of Tm during early larval development. Graph represents number of aggregates in Punc-54:polyQ40:yfp worms on different days of adulthood. Worms exposed to 0.125 μg/ml and 0.25 μg/ml Tm during larval development shows significant reduction in the number of aggregates on day 2 and day 3. Average percentage reduction over three biological replicates is plotted. Representative images shown in FIG. S5C. * represents p<0.05 as determined by student's t test, ns=non-significant.

FIG. S6: (A) The Punc-54:polyQ40:yfp or eat-2(ad1116;Punc-54:polyQ40:yfp worms were grown on control, ire-1 or sel-1 RNAi and western blot performed for SDS soluble PolyQ aggregates using anti-GFP antibody. β-actin was used to show equal loading of protein. (B) Representative images showing polyQ aggregates in Punc-54:polyQ40:yfp and ire-1(v33);Punc-54:polyQ40:yfp worms on control or drl-1 RNAi at day 2 of adulthood. Suppression of polyQ aggregation (indicated in %) on drl-1 knockdown is abrogated in ire-1 mutants. Average of three biological replicates is plotted. Error bars—Std. dev. (C) Representative images showing polyQ aggregates for Punc-54:polyQ40:yfp that has been treated transiently with Tm. Quantification is presented in FIG. 6C. (D) Graph represents reduction in the number of aggregates for Punc-54:polyQ40:yfp and ire-1(v33);Punc-54:polyQ40:yfp worms transiently exposed to 0.125 μg/ml Tm during hatching, as compared to untreated worms (Tm=0 μg/ml). Average of two biological replicates is plotted. Error bars—Std. dev. * represents p<0.05 as determined by Student's t test, ns=non-significant.

### DETAILED DESCRIPTION

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Throughout the description, the phrases “comprise” and “contain” and variations of them mean “including but not limited to”, and are not intended to exclude other moieties, additives, components, integers or steps. Thus, the singular encompasses the plural unless the context otherwise requires. Wherever there is an indefinite article used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification including any accompanying claims, abstract and drawings or any parts thereof, or to any novel one, or any novel combination, of the steps of any method or process so disclosed.

The following descriptions of particular embodiments and examples are offered by way of illustration and not by way of limitation.

Unless contraindicated or noted otherwise, throughout this specification, the terms “a” and “an” mean one or more, and the term “or” means and/or.

Unfolded protein response (UPR) of the endoplasmic reticulum (UPR^{ER}) helps maintain proteostasis in the cell. The ability to mount an effective UPR' to external stress (iUPR^{ER}) decreases with age and is linked to the pathophysiology of multiple age-related disorders. The inventors in the present disclosure have shown that a transient pharmacological ER stress, imposed early in development on Caenorhabditis elegans, enhances proteostasis, prevents iUPR^{ER} decline with age, and increases adult life span. Importantly, dietary restriction (DR), that has a conserved positive effect on life span, employs this mechanism of ER hormesis for longevity assurance.

The present inventors found that only the IRE-1-XBP-1 branch of UPR^{ER} is required for the longevity effects, resulting in increased ER-associated degradation (ERAD) gene expression and degradation of ER resident proteins during DR. Further, both ER hormesis and DR protect against polyglutamine aggregation in an IRE-1-dependent manner. It has been shown that the DR-specific FOXA transcription factor PHA-4 transcriptionally regulates the genes required for ER homeostasis and is required for ER preconditioning-induced life span extension. Together, the present invention provides a mechanism by which DR offers its benefits and opens the possibility of using ER-targeted pharmacological interventions to mimic the prolongevity effects of DR.

The endoplasmic reticulum (ER) deteriorates with age and fails to mount an effective stress response against misfolded proteins (UPR^{ER}), leading to protein folding disorders. However, preconditioning the ER using a mild ER stress (ER hormesis) can protect against future insults.

It has been shows that dietary restriction is an intervention that protects against protein misfolding disorders and increases life span across species, uses ER hormesis as a mechanism of action. Mimicking the ER hormesis in Caenorhabditis elegans by transient treatment with pharmacological reagent leads to delayed age-onset failure of UPR^{ER}, better capacity to process misfolded proteins, and increased life span.

In one embodiment, the present invention provides a method of inducing an early and transient upregulation of Unfolded Protein Response (UPRER) in a subject by either generating a nutrient restriction or administering pharmaceutical reagent, wherein said method mimics the pro-longevity effects of the dietary restriction (DR).

In another embodiment, said UPR^{ER} is upregulated in early stage of the life cycle of the subject.

In yet another embodiment, said UPR^{ER} is endoplasmic reticulum specific.

In an embodiment, the nutrient restriction is transient glucose deprivation.

In another embodiment, said pharmaceutical reagent is tunicamycin or 2-deoxyglucose.

In one embodiment, the present invention provides a method, wherein the mimicking effect of the dietary restrictions (DR) includes the following mechanism:
providing dietary restriction in early stage of the development of a subject for decreasing glycosylation of proteins that in turn leads to transient upregulation of the UPR^{ER} through conserved ER stress sensor IRE-1 and its downstream transcription factor XBP-1 and upregulation of ER-associated proteasomal degradation (ERAD) genes, wherein said upregulation leads to faster degradation of ER resident proteins as well as mis-folded and aggregated PolyQ proteins and thus reducing basal ER stress levels in the adult subject;
observing life span increase on transiently treated subjects.

In another embodiment, wherein the mimicking effect of the dietary restrictions (DR) increases the ability of the subject to mount an efficient UPR^{ER} during adulthood when challenged with an acute dose of tunicamycin or 2-deoxy glucose.

In yet another embodiment, the mimicking further includes a DR-specific transcription FOXA factor (PHA-4) for the transient UPRER during development as well as the upregulation of ERAD genes in adulthood in subjects undergoing DR.

In one embodiment, the present invention provides the method of treating protein mis-folding disorders and increasing the life span of a subject by applying the method as defined in the present specification.

In yet another embodiment, the present invention provide a pharmaceutical reagent or dietary restriction for inducing an early and transient upregulation of Unfolded Protein Response (UPR^{ER}) in a subject.

In an embodiment, the present invention provides use of pharmaceutical reagent or dietary restriction for inducing an early and transient upregulation of Unfolded Protein Response (UPR^{ER}) in a subject.

In a further embodiment, the present invention provides a kit claim comprising a pharmaceutical reagent and instruction manual for performing the method of inducing an early and transient upregulation of Unfolded Protein Response (UPR^{ER}) in a subject.

### EXAMPLES

The following examples serve to illustrate certain embodiments and aspects of the present disclosure and are not to be considered as limiting the scope thereof.

Material and Methods:

Detailed Experimental Procedures

C. elegans Strain Maintenance

Unless otherwise mentioned, all the C. elegans strains were maintained and propagated at 20° C. on E. coli OP50 using standard procedures. The strains used in this study were: N2 Bristol (wild-type), eat-2(ad1116)II, Phsp-4:gfp(zcIs4), rrf-3(pk1426)II, ire-1(v33)II, atf-6(ok551)X, pek-1(ok275)X, ire-1(ok799)II, xbp-1(zc12)III, xbp-1(tm2457)III, xbp-1(tm2482)III, hsp-4(gk514)II, Phsp-16.2;gfp(dvIS70), Phsp-6;gfp(zcIs9), ire-1(zc14)II;Phsp-4:gfp, rrf-3(pk1426)II, Punc-54:Q40:yfp(rmIs133). The above-mentioned strains were obtained from Caenorhabditis Genetics Centre, University of Minnesota, USA. The other strains including eat-2(ad1116)II;Phsp-4:gfp(zcIs4), eat-2(ad1116)II; rrf-3(pk1426)II, pek-1(ok275)X;Phsp-4:gfp(zcIs4), ire-1(v33)II;Punc-54:Q40:yfp(rmIs133) and eat-2(ad1116)II;Punc-54:Q40:yfp(rmIs133) were generated in-house using standard mating techniques.

Preparation of RNAi Plates

RNAi plates were poured using autoclaved (NGM) nematode growth medium supplemented with 100 μg/ml ampicillin and 2 mM IPTG. Plates were dried at room temperature for 1-2 days. Bacterial culture harbouring RNAi construct was grown in Luria Bertani (LB) media supplemented with 100 μg/ml ampicillin and 12.5 μg/ml tetracycline, overnight at 37° C. in a shaker incubator. Saturated cultures were re-inoculated the next day in fresh LB media containing 100 μg/ml ampicillin by using 1/100th volume of the primary inoculum and grown in 37° C. shaker until OD600 reached 0.5-0.6. The bacterial cells were pelleted down by centrifuging the culture at 5000 r.p.m for 10 minutes at 4° C. and resuspended in 1/10th volume of M9 buffer containing 100 μg/ml ampicillin and 1 mM IPTG. Around 350 μl of this resuspension was seeded onto RNAi plates and left at room temperature for 2 days for drying, followed by storage at 4° C. till further use.

Pellet from Part A was resuspended in 1×M9 buffer containing 100 μg/ml ampicillin and 1 mM IPTG to ½ of its volume (25 ml, if the initial culture volume of Part A was 50 ml). This resuspended bacterial culture was used as feed during the Tm treatment.

Hypochlorite Treatment to Obtain Eggs and Synchronizing Worm Population

Gravid adult worms, initially grown on E. coli OP50 bacteria were collected using M9 buffer in a 15 ml falcon tube. Worms were washed thrice by first centrifuging at 1800 r.p.m for 60 seconds followed by resuspension of the worm pellet in 1×M9 buffer. After the third wash, worm pellet was resuspended in 3.5 ml of 1×M9 buffer and 0.5 ml 5N NaOH and 1 ml of Sodium hypochlorite solution were added. The mixture was vortexed for 7-10 minutes until the entire worm bodies dissolved, leaving behind the eggs. The eggs were washed 5-6 times, by first centrifuging at 2500 r.p.m, decanting the 1×M9, followed by resuspension in 1×M9 buffer to remove traces of bleach and alkali. After the final wash, eggs were resuspended in approximately 100-200 μl of M9 and added to different RNAi plates

RNAi Life Span

Gravid adult worms, initially grown on E. coli OP50 were bleached and eggs were hatched on different RNAi plates. On reaching adulthood, 50-60 young adult worms were transferred to the similar RNAi plates in triplicates and overlaid with Fluoro-deoxyuridine (FudR) to final concentration of 0.1 mg/ml of agar (2). At the 7^{th} Day of adulthood, sick, sluggish and slow dwelling worms were removed from the life span population and the remaining were considered as the number of subjects ‘N’. Following this, number of dead worms were scored every alternate day and plotted as % survival against the number of days. Statistical analysis for survival was conducted using Mantel-Cox Log Rank test using Oasis software. Average life span was also determined using the same method and represented as Mean life span±Standard Error Mean (S.E.M).

Measurement of Basal UPR^{ER} During Larval Development

Transgenic worms expressing GFP under hsp-4 (mammalian ortholog GRP78/Bip) promoter [Phsp-4:gfp(zcIs4) and eat-2(ad1116);Phsp-4:gfp(zcIs4] were bleached and eggs were hatched on control RNAi. Fifty L3, L4, young adult or Day 1 gravid adult worms were immobilized on glass slides coated with 2% agarose using 20 mM sodium azide and visualized under Axio-imager M2 epifluorescent microscope (Carl Zeiss, Germany) equipped with a monochromatic camera lens (MRm) and GFP filter set. Fluorescence of ≥20 worms at different time points was quantified using NIH Image J software and represented as arbitrary units (AU). Basal UPRER in other strains like ire-1(zc14);Phsp-4:gfp(zcIs4) and on different RNAi like ire-1 or drl-1 was measured similarly.

Measurement of Induced UPR^{ER} Efficiency with Age

Transgenic Phsp-4:gfp(zcIs4) and eat-2(ad1116);Phsp-4:gfp(zcIs4) were bleached and eggs were hatched on control RNAi. Worms were allowed to grow till adulthood and then transferred onto plates overlaid with FuDR to a final concentration of 0.1 mg/ml. At each successive day (day 1 till day 4 of adulthood), approximately 100 worms were transferred to plates supplemented with 5 or 10 μg/ml tunicamycin (Tm) and incubated for 6 hours at 20° C. Since Tm is dissolved in 0.05% DMSO, it was used as vehicle control. After 6 hours, 50 worms from each treatment were mounted onto 2% agarose slides and visualized using Axio-Imager M2 epifluorescent microscope with a GFP filter set (Carl Zeiss, Germany) Fluorescence of ≥20 Tm-treated and untreated worms was quantified using NIH ImageJ software. Average fluorescence of treated worms was normalized to untreated worms and plotted as normalized GFP fold induction at different days of adulthood. Similar procedure was followed to compare iUPR^{ER} efficiency of Phsp-4:gfp(zcIs4) on control and drl-1 RNAi on different days of adulthood.

Tunicamycin Hormesis Treatment

Preparation of Feed:

Overnight grown bacterial culture expressing Control or test RNAi were re-inoculated in 200 ml fresh LB media and grown at 37° C. until OD600 reached 0.5-0.6. The culture was divided in two parts (A and B) in a ratio of 1:3 and cells are pelleted separately using a centrifuge (4810R, Eppendorf, Germany) at 5000 r.p.m for 10 minutes at 4° C. Pellet from part B (150 ml) of the culture was resuspended in 1×M9 buffer containing 100 μg/ml ampicillin and 1 mM IPTG to 1/10th of its initial culture volume (for 150 ml culture, 15 ml of resuspension buffer was used) and 350 μl was seeded onto each RNAi plate and left for drying at room temperature for 24 hours.

Preparation of Worms

Gravid adult worms initially grown on E. coli OP50 bacteria were bleached and eggs were re-suspended in 100-200 ul of 1×M9 buffer and standardized for the number of eggs present in a particular volume.

Preparation of the Cocktail

An intermediate stock solution of Tunicamycin (10 μg/ml) was prepared in MQ using a stock of 25 mg/ml (Sigma Aldrich, USA). In the final reaction mixture, this was diluted so as to treat the eggs with a variety of Tm concentrations ranging from 0.062 to 1 μg/ml. Entire composition of cocktail (total volume 1 ml) is mentioned as follows:

| | | Tm Volume | | |
|---|---|---|---|---|
| Tm | Feed | (10 μg/ml | Eggs | |
| Concentration | Volume | stock conc.) | volume | M9 |
| (μg/ml) | (μl) | (μl) | (μl) | (μl) |
| 0.125 | 875 | 12.5 | 25 | 87.5 |
| 0.25 | 875 | 25 | 25 | 75 |
| 0.5 | 875 | 50 | 25 | 50 |
| 1 | 875 | 100 | 25 | 0 |

Eggs were left on rotation in this cocktail at a slow speed for 24 hrs. Following this, the hatched L1 larvae were washed 3-4 times with 1×M9 buffer to remove traces of tunicamycin and added onto seeded control RNAi or test RNAi plates containing no reagent. They were allowed to grow till adulthood when 100 worms belonging to each treatment regime were transferred onto FuDR containing RNAi plates and life span was scored as mentioned above. iUPR^{ER} efficiency was also measured on Day 2 of adulthood, as mentioned previously.

DAVID Analysis

The RNA-seq data for synchronised Day1 adult worms [WT and eat-2(ad1116)] grown on E. coli OP50 (submitted to NCBI with the BioProject ID PRJNA342407) was analyzed using DAVID functional annotation tool. The listed pathways have p<0.05 and FDR<10 (Bonferroni Corrected p-value—0.0011).

ChIP-Seq Data Analysis

The ChIP-seq data analysis was performed using parameters mentioned in Singh A, et al. (2016) [A chromatin modifier integrates insulin/IGF-1 signalling and dietary restriction to regulate longevity. Aging Cell 15(4):694-705]. PHA-4 ChIP-seq data (SRA-NCBI GSE50301) was downloaded from modENCODE in .sra format. Downloaded data was converted into fastq format using NCBI-recommended SRA toolkit (version 2.2.2a). Converted fastq of replicates were merged and used for further analysis. Reads were aligned to the C. elegans genome (WS230) using Bowtie (v0.12.7) with the following parameters: -q -m 1 --best --strata. Mapped reads were used for peak calling and calculation of read density. Enriched peaks were identified using the peak calling algorithm MACS (v1.4.2) using following parameters: --mfold=5,30 --bw=175 -w. Statistically significant peaks (P<1×10-5) were used for further analysis. To find target genes, PeakAnalyzer (v1.4) program was used and all genes having peaks within 2 kb of the promoter region were considered for further analysis. UCSC genome browser was used for visualizing aligned data as wig files.

### Example 1

DR Triggers a Transient Up-Regulation of UPR^{ER} During Early Larval Development

In order to elucidate the kinetics of UPR^{ER} during nutrient stress, the levels of UPR^{ER} in the two genetic paradigms of DR was evaluated, namely eat-2(ad1116) and drl-1 RNAi. For this, Phsp-4:gfp(zcIs4) transgenic strain was used, where the promoter of hsp-4 was transcriptionally fused to gfp and GFP fluorescence gives a quantitative readout of UPR^{ER}. The GFP fluorescence in Phsp-4:gfp(zcIs4) and eat-2(ad1116);Phsp-4:gfp(zcIs4) at 32-35 hours post-bleaching was compared and it was found that DR triggered a transient up-regulation of UPR^{ER} specifically during the L2 larval stage of eat-2(ad1116) (FIG. 1A). The basal UPR^{ER} was found to be same in WT or DR worms in other larval stages, suggesting that only an early-life transient up-regulation of UPR^{ER} is specific to conditions that mimic DR (FIG. 1B). Likewise, Phsp-4:gfp(zcIs4) was grown on control or drl-1 RNAi and found that DR implemented by drl-1 KD triggered a similar transient up-regulation of UPR^{ER} specifically during the L2 larval stage (at 32 hours post-bleaching) (FIGS. S1A, S1B). Importantly, this response was found to be ER-specific as the cytosolic [Phsp-16.2:gfp(dvIs20)] or mitochondrial [Phsp-6:gfp(zcIs9)] stress response reporters were not affected (FIG. S1C). This early and transient up-regulation of the ER stress response was found to be dependent on the IRE-1 branch of UPR as eat-2(ad1116);Phsp-4:gfp(zcIs4) grown on ire-1 RNAi as well as the ire-1(zc14);Phsp-4:gfp(zcIs4) worms grown on drl-1 RNAi were unable to show similar response (FIGS. 1C, S1D). Together, an early and transiently high basal UPR^{ER} in eat-2(ad1116) mutants and on drl-1 knockdown indicates higher ER stress levels at initial larval life in worms undergoing a DR-like condition since hatching.

In the present invention media supplemented with 2% glucose was used and Phsp-4:gfp(zcIs4) or eat-2(ad1116);Phsp-4:gfp(zcIs4) worms are grown on the same. It was observed that the transient UPR^{ER} upregulation is mitigated on glucose-containing media in eat-2(ad1116) (FIG. 1D), suggesting that glucose deprivation may cause the early ER stress during DR. Glucose is essential for protein glycosylation and hence ER proteostasis; depletion could decrease protein glycosylation and increase ER-stress. Consistent with this, the inventors found that eat-2(ad1116) worms have lower levels of protein glycosylation compare to WT, using a Concavalin A (ConA) western assay (FIG. 1E). The level of ConA signal increases when the worms are supplemented with glucose. Together, DR triggers a transient upregulation of UPR^{ER}, possibly as a result of glucose restriction.

### Example 2

Life Span Extension by DR is Dependent on IRE-1

Knocking down the eat-2 gene or implementing BDR has been shown to decrease proteotoxicity and enhance longevity in adult worms. Since in higher eukaryotes, ER is responsible for folding around 70% of the proteome, the importance of the UPR^{ER} machinery in proteo-protective, longevity benefits conferred by DR has been investigated in the present invention. Towards this end, life span analysis was performed in two genetic mimics of DR, in the presence or absence of ire-1, atf-6 or pek-1, the ER membrane proteins that function to sense misfolded protein stress. Interestingly, knocking down ire-1 led to a significant suppression in life span of eat-2(ad1116) (FIG. 2A, Table S1) while the life span extension by drl-1 KD was completely abolished in an ire-1 mutant ire-1(v33) (FIG. S2A, Table S1). However, the other signal sensors, i.e. pek-1 and atf-6 were found to be dispensable for both the genetic paradigms of DR-mediated longevity (FIGS. 2B-C, S2B-C, Table S1). These data demonstrate that specific UPR^{ER} signalling through IRE-1 is required for eat-2 and drl-1 KD-mediated life span extension.

TABLE S1
Summary of life span analysis

| | | | | | | |
|---|---|---|---|---|---|---|
| Genetic | | | Mean ± SEM | % | | No of |
| Background | RNAi | N | (days) | Change | P-value | experiments |

FIG. 2A-C

| | | | | | | |
|---|---|---|---|---|---|---|
| rrf-3(pk1426) | control | 256 | 16.41 ± 0.21 | | | 4 |
| eat-2(ad1116):rrf- | control | 526 | 27.31 ± 0.15 | 66.42 | >0.0001 | 4 |
| 3(pk1426) | | | | | | |
| rrf-3(pk1426) | ire-1 | 248 | 16.69 ± 0.21 | | | 4 |
| eat-2(ad1116):rrf- | ire-1 | 329 | 21.67 ± 0.16 | 29.84 | >0.0001 | 4 |
| 3(pk1426) | | | | | | |
| rrf-3(pk1426) | pek-1 | 191 | 16.6 ± 0.24 | | | 4 |
| eat-2(ad1116):rrf- | pek-1 | 299 | 26.57 ± 0.21 | 60.06 | >0.0001 | 4 |
| 3(pk1426) | | | | | | |
| rrf-3(pk1426) | atf-6 | 187 | 16.78 ± 0.27 | | | 4 |
| eat-2(ad1116):rrf- | atf-6 | 319 | 25.77 ± 0.19 | 53.58 | >0.0001 | 4 |
| 3(pk1426) | | | | | | |

FIG. S2A-C

| | | | | | | |
|---|---|---|---|---|---|---|
| Wild type | Control | 350 | 20.51 ± 0.27 | | | 4 |
| | drl-1 | 201 | 27.54 ± 0.33 | 34.27 | >0.0001 | 4 |
| ire-1(v33) | Control | 413 | 19.45 ± 0.25 | | | 4 |
| | drl-1 | 475 | 18.60 ± 0.26 | −4.37 | 0.193 | 4 |
| pek-1(ok275) | Control | 228 | 21.21 ± 0.44 | | | 3 |
| | drl-1 | 147 | 27.96 ± 0.44 | 31.80 | >0.0001 | 3 |
| atf-6(ok551) | Control | 212 | 21.22 ± 0.36 | | | 3 |
| | drl-1 | 200 | 31.55 ± 0.40 | 48.60 | >0.0001 | 3 |

### Example 3

Early and Transient Up-Regulation of UPR^{ER} is Sufficient for Life Span Extension

Since transient up-regulation of UPR^{ER} was observed, experiments were conducted to see its causal role in life span extension, as observed during DR. For this, the transient UPR^{ER} up-regulation was mimicked through an external supplementation of a small dose of Tunicamycin (Tm) for 24 hrs during hatching of the eggs in liquid culture. After the pharmaceutical reagent was washed off, the worms were grown on solid NGM media and post-adult life span was recorded. Interestingly, it was found that exposing WT larvae to 0.125 μg/ml of Tm during the first 24 hours of its post-embryonic life led to significant extension of life span (FIG. 2D). Similar to DR, this longevity effect was completely dependent on ire-1(FIG. 2E). This observation supports the role of hormesis, triggered by an early ER stress as a mechanism of DR-mediated life span enhancement, in the models that were tested. Importantly, pharmacological ER stress could not further extend the life span of eat-2(ad1116) worms (FIG. 2F), suggesting that these two interventions use similar adaptive response to retard aging. Additionally, transiently supplementing non-hydrolysable glucose (2-deoxy glucose; 2DG) was also capable of significantly increasing life span, in an ire-1-dependent manner (FIGS. S2D, E). These results suggest that a hormetic dose of ER stress may be responsible for increased life span observed during DR.

TABLE S1
Summary of life span analysis
FIG. 2D-F

| | Tm | | | | | |
|---|---|---|---|---|---|---|
| Genetic | Conc | | Mean ± SEM | % | | No of |
| Background | (μg/ml) | N | (days) | Change | P-value | experiments |
| Wild type | 0 | 212 | 18.45 ± 0.23 | | | 3 |
| | 0.125 | 167 | 21.73 ± 0.28 | 17.78 | >0.0001 | 3 |
| Wild type (for ire-1 | 0 | 275 | 18.53 ± 0.22 | | | 3 |
| hormesis expt) | | | | | | |
| | 0.125 | 252 | 22.02 ± 0.30 | 18.83 | >0.0001 | 3 |
| ire-1(v33) | 0 | 310 | 18.80 ± 0.21 | | | 3 |
| | 0.125 | 266 | 18.64 ± 0.21 | −0.85 | 0.163 | 3 |
| Wild type (for eat-2 | 0 | 258 | 18.63 ± 0.24 | | | 2 |
| hormesis expt) | | | | | | |
| | 0.125 | 190 | 21.58 ± 0.36 | 15.83 | >0.0001 | 2 |
| eat-2 (ad1116) | 0 | 260 | 27.97 ± 0.36 | | | 2 |
| | 0.125 | 168 | 25.32 ± 0.45 | −10.47 | >0.0001 | 2 |

FIG. S2D-E

| | 2-DG | | | | | |
|---|---|---|---|---|---|---|
| Genetic | Conc | | Mean ± SEM | % | | No of |
| Background | (mM) | N | (days) | Change | P-value | experiments |
| Wild type | 0 | 158 | 18.99 ± 0.35 | | | 3 |
| | 0.0625 | 203 | 21.21 ± 0.37 | 11.69 | >0.0001 | 3 |
| ire-1(v33) | 0 | 264 | 20.20 ± 0.19 | | | 3 |
| | 0.0625 | 230 | 20.07 ± 0.26 | −0.64 | 0.4714 | 3 |

### Example 4

DR and a Hormetic Dose of ER Stress Delays the Age-Related Decline in iUPR^{ER} Efficiency

The efficiency of the ER stress response decreases with age, resulting in a progressively dysfunctional ER. Not only the levels of various UPR^{ER} target genes decrease in old rats and mice, the efficiency of mounting UPR^{ER} in response to various insults also decrease with age. To test whether the iUPR^{ER} efficiency in maintained in the genetic models of DR on different days of adulthood, this experiment was conducted. Also, a decline in iUPR^{ER} efficiency on the second and third day of adulthood in WT worms, respectively [FIGS. 3A (dark blue bars), S3A (dark green bars)] was observed. Importantly, this decline was significantly delayed in DR worms as both eat-2(ad1116);Phsp-4:gfp as well as Phsp-4:gfp worms grown on drl-1 RNAi displayed higher inductions of GFP fluorescence following Tm treatment [FIGS. 3A (light blue bars) and S3A (light green bars)]. The DR worms also maintained low basal levels of UPR^{ER} similar to that observed in daf-2(e 1370) (Henis-Korenblit et al., 2010) (FIGS. S3B, S3C).

Since it was observed that transient ER stress using Tₘ at early post-embryonic stage increased life span, it was tested whether this treatment could also lead to a better iUPR^{ER} efficiency with age. Towards this end, the iUPR^{ER} efficiency of transiently Tm-treated worms on day 2 of adulthood was checked and it was found that these worms displayed higher iUPR^{ER} efficiency (FIG. 3B). These experiments suggest that ER in DR worms are maintained in a healthier state and are more potent in combating external stressors even at a later age, when the WT ER efficiency typically declines. Importantly, hormetic up-regulation of the ER stress response during early developmental life leads to this increased ER efficiency contributing towards enhanced longevity during DR.

### Example 5

DR Increases the Expression of Genes Responsible for Protein Homeostasis within ER

The data obtained suggests that DR may help to partly combat the proteostasis collapse that occurs within the first two days of adult life, through a hormetic upregulation of UPR^{ER} during development. In an effort to better understand this process during DR, the changes in the global transcriptome profile of wild-type and eat-2(ad1116) at day 1 of adulthood was assessed). It was determined that the Gene-Ontology terms and KEGG pathways for the genes that were up regulated >2 folds (p-value≤0.05) in eat-2(ad1116) mutant as compared to WT and it was found that genes associated with protein folding functions within the ER were significantly enriched in this set (FIG. 4A). A total of 41 transcripts comprising of the proximal UPR sensors (ire-1, atf-6 and pek-1), hsp-70 family members, J-domain proteins and Endoplasmic Reticulum-Associated Degradation (ERAD) components were up-regulated (Table S2). Many of these genes are well-known UPR^{ER} targets. These results suggested that DR leads to a transcriptional reprogramming that supports a more efficient ER.

TABLE S2
List of ER protein processing genes up-regulated in eat-
2(ad1116) mutant as compared to WT at Day 1 of adulthood.

| | | Gene | Fold up- | | |
|---|---|---|---|---|---|
| No. | | name | regulation | P-value | Function |
| 1 | EPG | skr-5 | 2.67 | 0.050 | SKp1 related(ubiquitin ligase component |
| 2 | DNAJ | dnj-29 | 2.68 | >0.0001 | J domain protein |
| 3 | EPG | skr-8 | 2.01 | >0.0001 | SKp1 related(ubiquitin ligase component |
| 4 | DNAJ | dnj-7 | 3.16 | 0.002 | J domain protein |
| 5 | | ZK1307.8 | 5.17 | >0.0001 | predicted to have calcium ion binding activity |
| 6 | EPG | skr-13 | 3.58 | >0.0001 | SKp1 related(ubiquitin ligase component |
| 7 | EPG | skr-3 | 2.03 | >0.0001 | SKp1 related(ubiquitin ligase component |
| 8 | EPG | skr-9 | 2.53 | 0.006 | SKp1 related(ubiquitin ligase component |
| 9 | | sec-12 | 2.59 | >0.0001 | Prolactin regulatory element binding |
| 10 | | T24H7.2 | 4.7 | >0.0001 | Encodes an ortholog of hypoxia-upregulated |
| | | | | | vertebrate proteins, chaperone |
| 11 | EPG | skr-10 | 2.87 | >0.0001 | SKp1 related(ubiquitin ligase component |
| 12 | | atf-6 | 3.89 | 0.040 | proximal sensor required for theUPR, with a bZIP |
| | | | | | transcription factor domain |
| 13 | | T14G8.3 | 5.41 | >0.0001 | encodes an ortholog of hypoxia-upregulated |
| | | | | | vertebrate proteins, chaperone |
| 14 | | sec-24.1 | 2.57 | >0.0001 | a member of the Sec24-Sec23 subunit of the |
| | | | | | COPII coat complex |
| 15 | | C14B9.2 | 3.48 | >0.0001 | encodes a protein disulfide isomerase |
| 16 | | sec-24.2 | 3.05 | >0.0001 | a member of the Sec24-Sec23 subunit of the |
| | | | | | COPII coat complex |
| 17 | EPG | sel-1 | 3.5 | >0.0001 | member of the HRD complex that degrades |
| | | | | | malfolded ER proteins |
| 18 | HSP | F44E5.4 | 6.71 | 0.005 | hsp-70 family member |
| 19 | DNAJ | dnj-14 | 2.04 | 0.001 | J domain protein |
| 20 | | ero-1 | 2.72 | >0.0001 | Oxidoreductase in ER |
| 21 | EPG | ubxn-6 | 2.05 | >0.0001 | ubiquitin regulatory X domain-containing protein |
| 22 | DNAJ | dnj-12 | 2.4 | >0.0001 | J domain protein |
| 23 | HSP | F44E5.5 | 7.64 | >0.0001 | hsp-70 family member |
| 24 | EPG | cdc-48.3 | 3.5 | 0.001 | Final step in ubiquitin mediated proteolysis |
| 25 | EPG | F10C2.5 | 9.16 | >0.0001 | ortholog of human EDEM2 |
| 26 | | uggt-1 | 4.63 | >0.0001 | encodes UDP-Glc: glycoprotein |
| | | | | | glucosyltransferase (UGGT), an ER enzyme |
| | | | | | involved in quality control |
| 27 | EPG | Y105E8A.2 | 3.73 | >0.0001 | endoplasmic reticulum lectin 1 |
| 28 | EPG | C47E12.3 | 2.69 | 0.002 | ortholog of human EDEM1 (ER degradation |
| | | | | | enhancer, mannosidase alpha-like 1 |
| 29 | | pdr-1 | 2.44 | >0.0001 | parkin protein |
| 30 | EPG | ubc-15 | 2.23 | 0.003 | E2 ubiquitin-conjugating enzyme |
| 31 | EPG | ufd-2 | 4.8 | >0.0001 | E4 ubiquitin conjugation factor, in complex with |
| | | | | | cdc48 |
| 32 | EPG | ile-1 | 2.27 | >0.0001 | intracellular lectin |
| 33 | | pek-1 | 5.52 | >0.0001 | UPR sensor, orthologous to human eukaryotic |
| | | | | | translation initiation factor 2-alpha kinase 3 |
| 34 | HSP | hsp-70 | 3.05 | 0.002 | hsp-70 family member |
| 35 | HSP | F54F2.9 | 7.47 | >0.0001 | hsp-70 family member |
| 36 | EPG | ZC506.1 | 6.07 | 0.002 | ortholog of human EDEM3 |
| 37 | EPG | unc-23 | 4.79 | >0.0001 | negatively regulate proteosomal degradation by |
| | | | | | the E3 ubiquitin ligase |
| 38 | | cul-1 | 2.97 | >0.0001 | cell cycle protein |
| 39 | EPG | ire-1 | 3.75 | >0.0001 | proximal sensor, with an endoribonuclease |
| | | | | | activity |
| 40 | DNAJ | dnj-1 | 4.76 | >0.0001 | J domain protein |
| 41 | EPG | cnx-1 | 3.99 | >0.0001 | Calcium Binding chaperone, for glycoprotein |
| | | | | | folding |

EPG = ERAD pathway genes, HSP = hsp-70 proteins and DNAJ = DnaJ proteins

The quantitative real-time PCR (QRT-PCR) analysis was performed to validate the sequencing results. There was an increase in the mRNA levels in five of the UPR target genes; sel-1,sel-11, ufd-2, pdr-1 and dnj-1 in eat-2 mutant worms as compared to WT (FIG. 4B).

This increase in expression starts early in development in eat-2 mutant worms undergoing DR and is sustained till adulthood (FIG. S5A). Similarly, implementing DR by knocking down drl-1 also led to a significant increase in the mRNA level of these UPR target genes during adulthood, suggesting conserved up-regulation of this pathway in different DR paradigms (FIG. S5B). It was further identified that the upregulation of these UPR genes is dependent on the UPR sensor ire-1, in both the eat-2 and drl-1 paradigms (FIGS. S5C and D).

### Example 6

ERAD in DR Worms Efficiently Degrades ER Proteins

ERAD is an adaptive mechanism of the ER that regulates protein homeostasis within the organelle. It involves degradation of the misfolded proteins so as to prevent their accumulation inside the ER lumen. In the DR transcriptome mentioned above, genes pertaining to ERAD pathway formed almost half of the enriched ER gene set (19 out of 41 up-regulated transcripts) (FIG. 4C, Table S2). This indicated that DR in the two paradigms that they tested, may transcriptionally up-regulate ERAD, possibly to maintain proteostasis within the organelle at adulthood. To validate this, the inventors performed a cycloheximide chase assay in WT and eat-2(ad1116) worms on day 1 of adulthood to probe the turnover kinetics of ER-resident protein (FIG. 4D). Protein abundance at a particular time is a cumulative result of active protein synthesis and degradation. Cycloheximide is a protein synthesis inhibitor and supplementation of this reagent followed by western blotting allows for specifically reporting the rate of protein degradation. The levels of HSP-4, an ER resident protein were compared, after cycloheximide treatment in WT and eat-2 mutant and it was found a faster degradation of HSP-4 in DR worms (FIG. 4D). This indicates a more competent ERAD machinery operating within DR worms as compared to their wild-type counterpart. Proteins destined for degradation are affixed with a poly-ubiquitin chain, that serves as a degradation signal. Therefore, the changes in ubiquitination profile of WT and DR worms were assessed, by performing a western blot using a polyubiquitin antibody. Consistently with a robust ERAD, higher ubiquitination of proteins in eat-2(ad1116) was observed, suggesting that more proteins are destined for degradation under DR (FIG. 4E). Better ERAD is also indicative of a healthy ERAD which the DR worms may have that contributes to increased longevity.

### Example 7

Life Span Extension by DR is Dependent on ERAD Genes

Upon finding evidence of an up-regulated ERAD during DR, experiments were conducted to see whether it is required for promoting increased longevity. In this regard a life span analysis of WT and eat-2(ad1116) worms was performed on bacteria expressing RNAi against two of the up-regulated ERAD genes, ufd-2 and sel-1. SEL-1 (orthologue of mammalian HRD3) is a vital component of HRD-3/HRD-1 E3 ubiquitin ligase complex while UFD-2 functions in complex with CDC48 to regulate the length of polyubiquitin chains attached to proteins. It was found that life span extension of eat-2(ad1116) was partially suppressed on knocking down ufd-2 or sel-1 (FIG. 4F, Table S1), supporting their important role in DR mediated longevity. Altogether, DR promotes a healthy ER that can mount an effective ER stress response with age while efficiently degrading ER resident proteins to maintain ER homeostasis and increase life span.

TABLE S1
Summary of life span analysis
FIG. 4F

| Genetic | | | Mean ± SEM | % | | No of |
|---|---|---|---|---|---|---|
| Background | RNAi | N | (days) | Change | P-value | experiments |
| rrf-3(pk1426) | control | 256 | 16.41 ± 0.21 | | | 4 |
| eat-2(ad1116):rrf-3(pk1426) | control | 526 | 27.31 ± 0.15 | 66.42 | >0.0001 | 4 |
| rrf-3(pk1426) | ufd-2 | 177 | 17.12 ± 0.25 | | | 4 |
| eat-2(ad1116):rrf-3(pk1426) | ufd-2 | 342 | 24.12 ± 0.15 | 40.89 | >0.0001 | 4 |
| rrf-3(pk1426) (for sel-1 | control | 260 | 16.00 ± 0.20 | | | 3 |
| RNAi expt) | | | | | | |
| eat-2(ad1116):rrf- | control | 409 | 26.95 ± 0.18 | 68.44 | >0.0001 | 3 |
| 3(pk1426)(for sel-1 | | | | | | |
| RNAi expt) | | | | | | |
| rrf-3(pk1426) | sel-1 | 184 | 16.55 ± 0.24 | | | 3 |
| eat-2(ad1116):rrf-3(pk1426) | sel-1 | 280 | 22.10 ± 0.20 | 33.53 | >0.0001 | 3 |

### Example 8

FOXA Transcription Factor PHA-4 Controls Important Aspects of ER Homeostasis.

The C. elegans FOXA transcription factor PHA-4 is required for increased life span that is observed in multiple genetic as well as nongenetic paradigms of DR. Hence, the inventor tested whether up-regulation of the ER protein processing genes in an eat-2 mutant worm is dependent on PHA-4.

Using metadata analysis of published PHA-4 ChIP-seq data, it was found that PHA-4 binds to the promoter proximal sites of all of the 5 ER protein processing genes that are up-regulated in eat-2(ad1116) (FIG. 5A). QRT-PCR analysis showed that knocking down pha-4 significantly suppressed the induction of these genes in eat-2(ad1116) at young adult stage (FIG. 5B). This suggests that DR transcriptionally up-regulates genes that maintain protein folding homeostasis in the ER during early adulthood, in a manner dependent on ER sensor ire-1 and DR specific FOXA transcription factor PHA-4. Next, it was tested whether PHA-4/FOXA is involved in any other aspect of ER homeostasis. For that, we knocked down pha-4 using RNAi is hsp-4::gfp or eat-2(ad1116)::hsp-4::gfp and measured the transient up-regulation of ER stress early during development as seen in the genetic paradigms of DR. It was found that the eat-2(ad1116) worms lose the capability to mount the transient response at L2 stage when pha-4 is knocked down (FIG. 5C), while there was no effect of pha-4 RNAi on hsp-4::gfp in WT background. Next, it was tested whether PHA-4 regulates the iUPR^{ER} at adulthood when the worms are preconditioned at L2 with a hormetic dose of ER stress using Tm. Intriguingly, in absence of preconditioning, the adult pha-4 knockdown worms mounted an iUPR^{ER} (on acute 5 mg/mL tunicamycin treatment) (FIG. 5D; compare panels under 0 μg/mL column) that was much higher than the control RNAi worms. On the other hand, while the control RNAi worms mounted an even higher iUPR^{ER} when given Tm hormesis (compare panels in control RNAi row), pha-4 RNAi worms were not able to increase the iUPR^{ER} response, suggesting that the process is deregulated in absence of PHA-4. Consequently, knocking down PHA-4 suppressed the Tm hormesis- mediated life span extension (FIG. 5E; compare panels in control RNAi). Coupled to many other DR-specific functions of PHA-4, these experiments justify the important role of this transcription factor in DR-mediated life span extension.

### Example 9

DR Alleviates Polyglutamine Aggregation in an Ire-1 and ERAD-Dependent Manner

In the context of protein homeostasis, a better health-span will translate into fewer deleterious proteins accumulating or aggregating within the cell. Proteins that carry a stretch of polyglutamine residue over a threshold length are known to aggregate with age. These form the basis for various polyglutamine expansion disorders like Huntington's and Spinocerebellar ataxias. The exposed hydrophobic patches formed by multiple glutamine residues, typically more than 35, increases the chances of protein aggregation. Inefficient degradation of these proteins with increasing age exacerbates aggregation and associated diseases. Supporting this, is the observation that impaired ERAD and ER stress are primary events that augment polyglutamine toxicity. C. elegans is used as a model organism to study polyglutamine aggregation owing to the availability of fluorescent reporters that help in visualizing aggregate formation in different tissues, in real time.

In order to evaluate the role of DR in augmenting the ERAD and its physiological consequences, a reporter was used that expresses a stretch of 40 glutamines (Q40) tagged with a yellow fluorescent protein in C. elegans muscle. This reporter strain was crossed with eat-2(ad1116) mutant worms to generate worms carrying both the reporter transgene and the eat-2 mutation (hereafter, referred to as eat-2;PolyQ). It was observed that incorporating eat-2 mutation led to 60-80% suppression in the number of aggregates at all age points, supporting the earlier observation of alleviation in the age-associated increase in PolyQ aggregation during DR (FIG. 6A). Knocking down drl-1 also decreased the number of aggregates observed in the reporter worm with age, suggesting a conserved phenomenon of DR-mediated reduction in aggregation (FIG. S6A). Since ire-1 was a requirement for DR-mediated longevity, it was tested whether this beneficial effect conferred under DR is also dependent on this gene. Consistently, it was observed that the percentage suppression of PolyQ aggregation observed in eat-2 mutant worms was significantly reduced on ire-1 knockdown (FIG. 6A). Similarly, ire-1 mutants failed to suppress the number of PolyQ aggregates on drl-1 knockdown at different days of adulthood, suggesting a dependence on ire-1 branch of UPR^{ER} for maintenance of proteostasis (FIG. S6A).

Finally, to further evaluate the role of ERAD machinery in DR-induced suppression of polyglutamine aggregation, eat-2;PolyQ worms were grown on control, ufd-2 or sel-1 RNAi. it was found that eat-2(ad1116) worms grown on ERAD gene RNAi had significantly less suppression of PolyQ puncta compared to control RNAi-grown worms (FIG. 6B).

This suggests that DR leads to a healthier ER that reduces polyglutamine aggregation in adult worms in a manner dependent on ire-1 and ERAD.

### Example 10

A Hormetic Dose of ER Stress, During Early Life, Up-Regulates ERAD and Suppresses polyQ Aggregation

It was reported that an atypical up-regulation of UPR^{ER} in larvae contributes towards increased life span during DR. It was shown that pharmaceutically mimicking the transient UPR^{ER} up-regulation during early larval life augmented the iUPR^{ER} efficiency with age and increased life span. To determine whether such hormetic intervention can protect against the age-related proteostasis collapse, WT worms were exposed with a range of concentrations of Tm only during the start of larval development, as mentioned before. The untreated and Tm-treated PolyQ:yfp worms were compared for the number of aggregates on different days of adulthood and witnessed a suppression in the treated worms (FIGS. 6C, S6C). Moreover, this phenomenon was also found to be dependent on the proximal UPR sensor ire-1, as ire-1(v33);Q40:yfp worms treated with a hormetic dose of Tm had significantly higher puncta at all age points (FIG. S6B).

1. A method of inducing an early and transient upregulation of Unfolded Protein Response (UPR^{ER}) in a subject by either generating a nutrient restriction or administering pharmaceutical reagent, wherein said method mimics the pro-longevity effects of the dietary restriction (DR).

2. The method as claimed in claim 1, wherein said UPR^{ER} is upregulated in early stage of the life cycle of the subject.

3. The method as claimed in claim 1, wherein said UPR^{ER} is endoplasmic reticulum specific.

4. The method as claimed in claim 1, wherein the nutrient restriction is transient glucose deprivation.

5. The method as claimed in claim 1, wherein said pharmaceutical reagent is tunicamycin or 2-deoxy glucose.

6. The method as claimed in claim 1, wherein the mimicking effect of the dietary restrictions (DR) includes the following mechanism:
providing dietary restriction in early stage of the development of a subject for decreasing glycosylation of proteins that in turn leads to transient upregulation of the UPR^{ER} through conserved ER stress sensor IRE-1 and its downstream transcription factor XBP-1 and upregulation of ER-associated proteasomal degradation (ERAD) genes, wherein said upregulation leads to faster degradation of ER resident proteins as well as mis-folded and aggregated PolyQ proteins and thus reducing basal ER stress levels in the adult subject;
observing life span increase on transiently treated subjects.

7. The method as claimed in claim 6, wherein the mimicking effect of the dietary restrictions (DR) increases the ability of the subject to mount an efficient UPR^{ER} during adulthood when challenged with an acute dose of tunicamycin or 2-deoxy glucose.

8. The method as claimed in claim 6, wherein the mimicking further includes a DR-specific transcription FOXA factor (PHA-4) for the transient UPR^{ER} during development as well as the upregulation of ERAD genes in adulthood in subjects undergoing DR.

9. A method of treating protein mis-folding disorders and increasing the life span of a subject by applying the method as claimed in claim 1.

10. A pharmaceutical reagent or dietary restriction for inducing an early and transient upregulation of Unfolded Protein Response (UPR^{ER}) in a subject.

11. A method of applying a pharmaceutical reagent or dietary restriction for inducing an early and transient upregulation of Unfolded Protein Response (UPR^{ER}) in a subject.

12. A kit claim comprising a pharmaceutical reagent and instruction manual for performing the method of inducing an early and transient upregulation of Unfolded Protein Response (UPR^{ER}) in a subject.
